(19) United States
(12) Patent Application Publication
(10) Pub. No.: US 2023/0248000 A1
(43) Pub. Date: Aug. 10, 2023

(54) ANTIMICROBIAL COMPOUNDS AND METHODS OF USE

(71) Applicant: Aequor, Inc., Encinitas, CA (US)

(72) Inventor: Cynthia K. Burzell, Encinitas, CA (US)

(73) Assignee: Aequor, Inc., Encinitas, CA (US)

(21) Appl. No.: 18/063,911

(22) Filed: Dec. 9, 2022

Related U.S. Application Data
(63) Continuation of application No. 16/310,274, filed on Dec. 14, 2018, now abandoned.
(60) Provisional application No. 62/301,488, filed on Feb. 29, 2016.

(51) Int. Cl. A01N 57/26; A01N 41/04; A01N 57/12; A01N 57/20; A01N 59/26; B63B 59/04; A61K 31/255

(52) U.S. Cl. CPC A01N 57/26; A01N 41/04; A01N 57/12; A01N 57/20; A01N 59/26; B63B 59/04; A61K 31/255

(57) ABSTRACT

Methods of using organophosphorus or organosulfur compounds to disperse, remove, or inhibit the growth of a biofilm, or inhibit the growth of, or kill a fungus or bacteria are provided.

### CROSS-REFERENCE TO RELATED APPLICATION

This application is a continuation of co-pending U.S. application Ser. No. 16/310,274 filed Dec. 14, 2018, which is a U.S. National Stage application of International Application No. PCT/US2017/020016, filed Feb. 28, 2017, which claims the benefit of U.S. Provisional Application No. 62/301,488, filed Feb. 29, 2016, the disclosures of each of which are explicitly incorporated herein by reference in their entirety.

### BACKGROUND OF THE DISCLOSURE

Microbial biofilms cause systemic infections in plants and animals, including humans, and cause costly marine and industrial related damage and inefficiency. They cost billions of dollars yearly in equipment damage, product contamination, energy losses and medical infections.

All living and non-living surfaces are potential sites for microbial biofilm formation. In the human body biofilms can be associated with tissues (e.g., inner ears, teeth, gums, lungs, heart valves and the urogenital tract) and on indwelling medical devices (e.g., contact lenses, central venous catheters and needleless connectors, endotracheal tubes, intrauterine devices, mechanical heart valves, pacemakers, peritoneal dialysis catheters, prosthetic joints, tympanostomy tubes, urinary catheters, and voice prostheses). An estimated 80% of all microbial infections involve biofilms.

Biofilms are a problem in the water service utilities and many industrial processes including the food, pharmaceutical, paint, oil and gas, and pulp and paper processing, manufacturing, and engineering industries. Biofilms also cause corrosion, scale, and slime in industrial systems, oil souring and biofouling. The attachment of marine foulers such as barnacles, mussels, algae, and other biofouling of ships' hulls is a major problem for shipping worldwide, causing hydrodynamic drag that requires over 50% additional fuel consumption to overcome, with corresponding increased noxious emissions.

Biofilms are a problem in consumer products including, cleaning products, soaps, lotions, cosmetics, etc. Biofilms result in contamination of the product by microorganisms resistant to the preservatives commonly used in cosmetics and personal care products. Biofilm contamination can occur at the manufacturing plant or after the product enters the consumers home.

Biofilms are extremely difficult to remove with existing technology because they can withstand high temperature (>150° C.), biocides, anti-infective compounds including antibiotics, and host immune responses. Also, the huge doses of antimicrobials required to rid systems of biofilm bacteria are environmentally undesirable and medically impractical. The overuse of biocides and antibiotics has triggered the emergence of antimicrobial resistant strains, all of which are biofilm-formers (e.g., MRSA [methicillin-resistant Staphylococcus aureus). Thus, there is an immediate need for safe and effective products that combat biofilms.

### BRIEF DESCRIPTION OF THE DRAWINGS

FIG. 1 depicts the average Minimum Inhibitory Concentrations (MICs) of 18 organophosphorus and organosulfurous representative compounds against Gram-positive and Gram-negative bacteria and fungi.

FIG. 2 depicts the average Minimum Biofilm Eradicating Concentrations (MBECs) of 15 organophosphorous and organosulfurous representative compounds against Gram-positive and Gram-negative bacteria.

### SUMMARY

The present disclosure is directed to methods of using organophosphorus or organosulfur compounds, and compositions comprising the compounds, as biofilm dispersants, biofilm removers, antibiofilm, antifouling, antimicrobial, and anti-fungal agents.

In one aspect, the invention is directed to methods of using as a biofilm dispersant, biofilm remover, antibiofilm, antifouling, antimicrobial, and/or anti-fungal agent, a compound represented by Formula I:

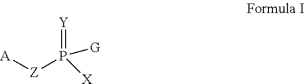

or a salt thereof; wherein A is H, C₁₋₁₆ hydrocarbyl or optionally R¹ substituted C₁₋₁₆ hydrocarbyl; Z is O or a bond; Y is O; G is OH, H, C₁₋₆—COO-alkyl, —O—C₁₋₁₆ alkyl, R¹ substituted C₁₋₁₀ hydrocarbyl, CH₂NHCH₂COOH, or O-aryl, X is H, CN, —NHR, —NHOR, —NHOCOR, R¹ substituted C₁₋₁₀ hydrocarbyl or —OR; and R is H, C₁₋₁₀ hydrocarbyl, or optionally R¹ substituted C₁₋₁₀ hydrocarbyl;

R¹ is selected from halogen, cyano, OH, C₁₋₆ hydrocarbyl, C₁₋₆ alkoxy; SOR², SO₂R², SO₂NR³R⁴, CONR³R⁴, NR³R⁴, NR³COR⁴, NR³SO₂R⁴, NR³CO₂R⁴, NR³CONR⁴, and phosphonic add, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy; cyano, or phosphonic acid;

R², R³ and R⁴ are independently selected from hydrogen, C₁₋₆ hydrocarbyl, in which each of the C₁₋₆ hydrocarbyl can be optionally substituted with halo, amino, hydroxyl, C₁₋₆ alkoxy, cyano, or phosphonic acid.

The methods of the present disclosure are methods of dispersing, removing, or inhibiting the growth of a biofilm, or inhibiting the growth of, or killing a fungus or bacteria, comprising contacting the biofilm, fungus or bacteria with an amount of a compound of Formula I, or a composition comprising an amount of a compound of Formula I, wherein the amount of the compound of Formula I is effective to disperse, remove or inhibit the growth of the biofilm, or inhibit the growth of, or kill the fungus or bacteria.

In some embodiments, the methods are methods of dispersing, removing, or inhibiting the growth of a biofilm comprising contacting the biofilm with an amount of a compound of Formula I, or a composition comprising an amount of a compound of Formula I, wherein the amount of the compound of Formula I is effective to disperse, remove, or inhibit the growth of the biofilm.

In some embodiments, the methods comprise contacting the biofilm with an amount of a compound of Formula I effective to disperse, remove, or inhibit the growth of the biofilm.

In some embodiments, the methods comprise contacting the biofilm with a composition comprising an amount of a compound of Formula I effective to disperse, remove, or inhibit the growth of the biofilm.

In some embodiments, the methods are methods of inhibiting the growth of, or killing a fungus or bacteria, comprising contacting the fungus or bacteria with an amount of a compound of Formula I, or a composition comprising an amount of a compound of Formula I, wherein the amount of the compound of Formula I is effective to inhibit the growth of or kill the fungus or bacteria.

In some embodiments, the methods comprise contacting the fungus or bacteria with an amount of a compound of Formula I effective to inhibit the growth of or kill the fungus or bacteria.

In some embodiments, the methods comprise contacting the fungus or bacteria with a composition comprising an amount of a compound of Formula I effective to inhibit the growth of or kill the fungus or bacteria.

In one aspect, the invention is directed to methods of using as a biofilm dispersant, biofilm remover, antibiofilm, antifouling, antimicrobial, and/or anti-fungal agent, a compound represented by Formula II:

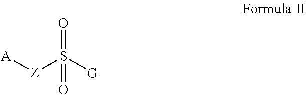

or a salt thereof; wherein A is H, C₁₋₂₀ hydrocarbyl, alkylaryl, or optionally R¹ substituted C₁₋₂₀ hydrocarbyl; Z is O or a bond; G is OH, H, C₁₋₆—COO-alkyl, —O—C₁₋₆ alkyl, R¹ substituted C₁₋₁₀ hydrocarbyl, CH₂NHCH₂COOH, or O-aryl;

R¹ is selected from halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, and phosphonic add, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, d-6 hydrocarbyl, C₁₋₆ alkoxy, cyano, or phosphonic acid;

R², R³ and R⁴ are independently selected from hydrogen, C₁₋₆ hydrocarbyl, in which each of the C₁₋₆ hydrocarbyl can be optionally substituted with halo, amino, hydroxyl, alkoxy, cyano, or phosphonic acid.

The methods of the present disclosure are methods of dispersing, removing or inhibiting the growth of a biofilm, or inhibiting the growth of, or killing a fungus or bacteria, comprising contacting the biofilm, fungus or bacteria with an amount of a compound of Formula II, or a composition comprising an amount of a compound of Formula II, wherein the amount of the compound of Formula II is effective to disperse, remove or inhibit the growth of the biofilm, or inhibit the growth of, or kill the fungus or bacteria.

In some embodiments, the methods are methods of dispersing, removing, or inhibiting the growth of a biofilm comprising contacting the biofilm with an amount of a compound of Formula II, or a composition comprising an amount of a compound of Formula II, wherein the amount of the compound of Formula II is effective to disperse, remove, or inhibit the growth of the biofilm.

In some embodiments, the methods comprise contacting the biofilm with an amount of a compound of Formula II effective to disperse, remove, or inhibit the growth of the biofilm.

In some embodiments, the methods comprise contacting the biofilm with a composition comprising an amount of a compound of Formula II effective to disperse, remove, or inhibit the growth of the biofilm.

In some embodiments, the methods are methods of inhibiting the growth of, or killing a fungus or bacteria, comprising contacting the fungus or bacteria with an amount of a compound of Formula II, or a composition comprising an amount of a compound of Formula II, wherein the amount of the compound of Formula II is effective to inhibit the growth of or kill the fungus or bacteria.

In some embodiments, the methods comprise contacting the fungus or bacteria with an amount of a compound of Formula II effective to inhibit the growth of or kill the fungus or bacteria.

In some embodiments, the methods comprise contacting the fungus or bacteria with a composition comprising an amount of a compound of Formula II effective to inhibit the growth of or kill the fungus or bacteria.

The compounds of Formula I or Formula II or compositions comprising compounds of Formula I or Formula II may be used for reducing or preventing fungal or microbial growth, such as growth of a bacteria or a biofilm, for dispersing or removing biofilms, or as antifouling agents.

Some embodiments include a method of reducing or preventing fungal or microbial growth on a surface comprising applying a an effective amount of a compound of Formula I or Formula II, or a composition comprising a compound of Formula I or Formula II to a surface, such as a surface susceptible to fungal or microbial growth or biofilm formation.

In some embodiments, the surface is a living surface, such as human, animal, or plant tissue. In some embodiments, the surface is a non-living surface, such as indwelling medical devices, or surfaces exposed to water, such as industrial equipment and marine vessels, or the surface of water itself.

### DETAILED DESCRIPTION

Unless specifically defined herein, all terms used herein have the same meaning as they would to one skilled in the art of the present disclosure.

As used herein, the term “antimicrobial” refers to an agent that is effective against pathogenic microorganisms, including bacteria, fungi, viruses, protozoa, and biofilms. Antimicrobial agents can be used to disperse, remove, inhibit, reduce, or prevent fungal or microbial growth.

The term “heteroatom” refers to any atom other than carbon, for example, N, O, or S.

The term “substituents” refers to groups such as hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, cyano, nitre, amino, amide, aldehyde, acyl, oxyacyl, carboxyl, sulfonyl, sulfonamide, sulfuryl, and the like.

The term “hydrocarbyl” refers to univalent groups formed by removing a hydrogen atom from a hydrocarbon, e.g. alkyl, cycloalkyl, alkenyl, alkynyl, aryl, alkylaryl, arylalkyl, arylalkenyl, arylalkynyl, and arylene. The term “substituted hydrocarbyl” refers to hydrocarbyl groups further bearing one or more substituents as defined herein.

The term “alkyl” refers to a monovalent straight or branched chain hydrocarbon group having from one to about 12 carbon atoms, for example, methyl, ethyl, n-propyl, iso-propyl, n-butyl, iso-butyl, tert-butyl, n-pentyl (also known as n-amyl), n-hexyl, and the like. The term “substituted alkyl” refers to alkyl groups further bearing one or more substituents as defined herein.

The term “alkenyl” refers to straight-chained or branched hydrocarbyl groups having at least one carbon-carbon double bond, and having between about 2 and about 12 carbon atoms, and the term “substituted alkenyl” refers to alkenyl groups further bearing one or more substituents as defined herein.

The term “alkynyl” refers to straight-chained or branched hydrocarbyl groups having at least one carbon-carbon triple bond, and having between about 2 and about 12 carbon atoms, and the term “substituted alkynyl” refers to alkynyl groups further bearing one or more substituents as defined herein.

The term “alkoxy” refers to the moiety —O-alkyl, wherein alkyl is as defined above, and the term “substituted alkoxy” refers to alkoxy groups further bearing one or more substituents as defined herein.

The term “cycloalkyl” refers to alkyl groups having between 3 and about 8 carbon atoms arranged as a ring, and the term “substituted cycloalkyl” refers to cycloalkyl groups further bearing one or more substituents as defined herein.

The term “aromatic” refers to a cyclically conjugated molecular entity with a stability, due to delocalization, significantly greater than that of a hypothetical localized structure, such as the Kekule structure.

The term “heterocyclic,” when used to describe an aromatic ring, refers to the aromatic rings containing at least one heteroatom, as defined above. The term “heterocyclic,” when not used to describe an aromatic ring, refers to cyclic (i.e., ring-containing) groups other than aromatic groups, the cyclic group being formed by between 3 and about 14 carbon atoms and at least one heteroatom as defined herein.

The term “substituted heterocyclic” refers, for both aromatic and non-aromatic structures, to heterocyclic groups further bearing one or more substituents as defined herein.

The term “aryl” refers to aromatic groups having between about 5 and about 14 carbon atoms and the term “substituted aryl” refers to aryl groups further bearing one or more substituents as defined herein.

The term “heteroaryl” refers to aromatic rings, where the ring structure is formed by between 3 and about 14 carbon atoms and by at least one heteroatom described above, and the term “substituted heteroaryl” refers to heteroaryl groups further bearing one or more substituents as defined herein.

The term “alkylaryl” refers to alkyl-substituted aryl groups and the term “substituted alkylaryl” refers to alkylaryl groups further bearing one or more substituents as defined herein.

The term “arylalkyl” refers to aryl-substituted alkyl groups and the term “substituted arylalkyl” refers to arylalkyl groups further bearing one or more substituents as defined herein.

The term “arylalkenyl” refers to aryl-substituted alkenyl groups and the term “substituted arylalkenyl” refers to arylalkenyl groups further bearing one or more substituents as defined herein.

The term “arylalkynyl” refers to aryl-substituted alkynyl groups and the term “substituted arylalkynyl” refers to arylalkynyl groups further bearing one or more substituents as defined herein.

The term “arylene” refers to divalent aromatic groups having between 5 and about 14 carbon atoms and the term “substituted arylene” refers to arylene groups further bearing one or more substituents as defined herein.

The present disclosure is directed to methods of using organophosphorus or organosulfur compounds, and compositions comprising the compounds, as biofilm dispersants, biofilm removers, antibiofilm, antifouling, antimicrobial, and/or anti-fungal agents.

In one aspect, the invention is directed to methods of using as a biofilm dispersant, biofilm remover, antibiofilm, antifouling, antimicrobial, and/or anti-fungal agent, a compound represented by Formula I:

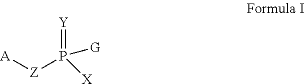

or a salt thereof; wherein A is H, C₁₋₁₆ hydrocarbyl or optionally R¹ substituted C₁₋₁₆ hydrocarbyl; Z is O or a bond; Y is O; G is OH, H, C₁₋₆—COO-alkyl, —O—C₁₋₁₆ alkyl, R¹ substituted C₁₋₁₀ hydrocarbyl, CH₂NHCH₂COOH, or O-aryl; X is H, ON, —NHR, —NHOR, —NHOCOR, R¹ substituted C₁₋₁₀ hydrocarbyl or —OR; and R is H, hydrocarbyl, or optionally R¹ substituted C₁₋₁₀ hydrocarbyl;

R¹ is selected from halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, and phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, cyano, or phosphonic add;

R², R³ and R⁴ are independently selected from hydrogen, C₁₋₆ hydrocarbyl, in which each of the C₁₋₆ hydrocarbyl can be optionally substituted with halo, amino, hydroxyl, C₁₋₆ alkoxy, cyano, or phosphonic add.

In one aspect, the invention is directed to methods of using as a biofilm dispersant, biofilm remover, antibiofilm, antifouling, antimicrobial, and/or anti-fungal agent, a compound represented by Formula II:

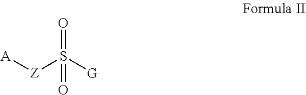

or a salt thereof; wherein A is H, C₁₋₂₀ hydrocarbyl, alkylaryl, or optionally R′ substituted

C₁₋₂₀ hydrocarbyl; Z is O or a bond; G is OH, H, C₁₋₆—COO-alkyl, —O—C₁₋₆ alkyl, R¹ substituted C₁₋₁₀ hydrocarbyl, CH₂NHCH₂COOH, or O-aryl;

R¹ is selected from halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, and phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, alkoxy, cyano, or phosphonic acid;

R², R³ and R⁴ are independently selected from hydrogen, C₁₋₆ hydrocarbyl, in which each of the C₁₋₆ hydrocarbyl can be optionally substituted with halo, amino, hydroxyl, C₁₋₆ alkoxy, cyano, or phosphonic acid.

With respect to any relevant structural representation, such as Formula I or Formula II, Z is O or a bond. In some embodiments, Z is O. In some embodiments, Z is a bond.

With respect to any relevant structural representation, such as Formula I, Y is O.

With respect to any relevant structural representation, such as Formula I or Formula II, G is OH, —O-alkyl (such as —OCH₃, —OC₂H₅, —OC₃H₇, —OC₄H₉, —OC₅H₁₁, —OC₆H₁₃, etc.), C₁₋₆—COO-alkyl, R¹ substituted C₁₋₁₀ hydrocarbyl, or O-aryl. In some embodiments, G is —COH₃. In some embodiments, G is —OC₂H₅. In some embodiments, G is —OC₄H₉. In some embodiments, G is CH₂COOCH₃. In some embodiments, G is OCH₂CF₃. In some embodiments, G is C₂H₄CHNH₂COOH. In some embodiments, G is O-phenyl. In some embodiments, G is CH₂NHCH₂COOH. In some embodiments, G is O Na.

With respect to any relevant structural representation, such as Formula I, X is H, CN, —NHR, —NHOR, —NHOCOR, R¹ substituted C₁₋₁₀ hydrocarbyl or —OR; and R is H, C₁₋₁₀ hydrocarbyl, or optionally R¹ substituted C₁₋₁₀ hydrocarbyl. In some embodiments, X is H. In some embodiments, X is OR. In some embodiments, X is OH. In some embodiments, X is OCH₃. In some embodiments, X is —OC₂H₅. In some embodiments, X is —NHR. In some embodiments; X is —NH₂. In some embodiments, X is OCH₂CF₃. In some embodiments X is CN.

With respect to any relevant structural representation, such as Formula I, R is H or C₁₋₁₀ hydrocarbyl, including C₁₋₁₀ alkyl (e.g. methyl; C₂ alkyl, such as ethyl; C₃ alkyl, such as propyl, isopropyl, cyclopropyl, etc.; C₄ alkyl, such as linear, branched or cyclic, butyl; etc.; C₅ alkyl, C₆ alkyl; C₇ alkyl, C₈ alkyl, C₉ alkyl, or Ow alkyl), C₁₋₆ alkyl, C₁₋₃ alkyl, CMO alkenyl (e.g. C₂ alkenyl, such as vinyl; C₃ alkenyl, such as —CH₂—CH═CH₂, C₄ alkenyl, such as linear, branched or cyclic, butenyl, etc.; C₅ alkenyl, C₆ alkenyl, C₇ alkenyl, C₈ alkenyl, C₉ alkenyl; or C₁₀ alkenyl), C₂₋₆ alkenyl, C₂₋₄ alkenyl, optionally substituted aryl, such as phenyl or hydrocarbyl substituted phenyl, naphthyl, etc. In some embodiments, R is H. In some embodiments, R is C₁₋₆ alkyl. In some embodiments, R is C₁₋₃ alkyl. In some embodiments, R is CH₃. In some embodiments, R is C₂H₅. In some embodiments, R is CH₂CF₃.

With respect to any relevant structural representation, such as Formula I or Formula II, A is H, C₁₋₂₀ hydrocarbyl, including C₁₋₁₂ alkyl (e.g. methyl; C₂ alkyl, such as ethyl; C₃ alkyl, such as propyl; isopropyl, cyclopropyl, etc.; C₄ alkyl, such as linear, branched or cyclic, butyl; etc.; C₅ alkyl, C₆ alkyl, C₇ alkyl, C₈ alkyl, C₉ alkyl, C₁₀ alkyl, C₁₁ alkyl or C₁₂ alkyl), C₁₋₆ alkyl, C₁₋₃ alkyl, C₁₋₁₀ alkenyl (e.g. C₂ alkenyl, such as vinyl; C₃ alkenyl, such as —CH₂—CH═CH₂, C₄ alkenyl, such as linear, branched or cyclic, butenyl; etc.; C₅ alkenyl (such as isopentenyl), C₆ alkenyl, C₇ alkenyl; C₈ alkenyl; C₉ alkenyl, or C₁₀ alkenyl), C₂₋₆ alkenyl, C₂₋₄ alkenyl, aryl, such as phenyl or napthyl, alkylaryl, or optionally R¹ substituted C₁₋₂₀ hydrocarbyl.

R¹ is selected from halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, and phosphonic acid, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, cyano, or phosphonic acid;

R², R³ and R⁴ are independently selected from hydrogen; C₁₋₆ hydrocarbyl, in which each of the C₁₋₆ hydrocarbyl can be optionally substituted with halo, amino, hydroxyl, C₁₋₆ alkoxy, cyano, or phosphonic acid.

With respect to any relevant structural representation, such as Formula I or Formula II, in some embodiments, A is H. In some embodiments, A is C₁₋₆ alkyl. In some embodiments, A is ethyl. In some embodiments, A is C₃ alkyl, such as n-propyl, isopropyl, or cyclopropyl. In some embodiments, A is n-propyl. In some embodiments, A is isopropyl. In some embodiments, A is C₄ alkyl, such as n-butyl, t-butyl, or cyclobutyl. In some embodiments, A is n-butyl. In some embodiments, A is t-butyl. In some embodiments, A is C₅ alky, such as n-pentyl, isopentyl, cyclopentyl, etc. In some embodiments, A is n-pentyl. In some embodiments, A is isopentyl. In some embodiments, A is C₆ alkyl, such as n-hexyl, cyclohexyl, etc. In some embodiments, A is n-hexyl. In some embodiments, A is C₃₋₅ alkenyl, such as propenyl, butenyl, isopentenyl, pentenyl, etc. In some embodiments, A is C₅ alkenyl. In some embodiments, A is isopentenyl. In some embodiments, A is alkylaryl.

With respect to any relevant structural representation, such as Formula I or Formula H, in some embodiments, A is —(CH₂)₁₋₂-Cy, wherein Cy is optionally substituted cycloalkyl (such as cyclopropyl, cyclobutyl, cyclopentyl, cyclohexyl) or optionally substituted phenyl. In some embodiments, A is

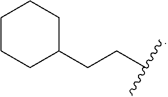 [SMILES: *CCC1CCCCC1]

In some embodiments, A is

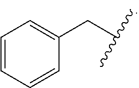 [SMILES: *Cc1ccccc1]

In some embodiments, A is R¹ substituted alkyl. In some embodiments, A is CF₃. In some embodiments, A is C₂H₄OH. In some embodiments, A is 1-amino-1-phenyl methyl. In some embodiments, A is 1-amino-2-phenyl-ethyl.

In some embodiments of Formula I, Y is O, Z is O, A is C₄H₉, G is OC₄H₉, and X is selected from the group consisting of H, OH, OCH₃, and NH₂. In some embodiments, X is H. In some embodiments, X is OH. In some embodiments, X is OCH₃. In some embodiments, X is NH₂.

In some embodiments of Formula I, Y is O, Z is O, A is CH₂CF₃, G is CH₂COOCH₃, and X is OCH₂CF₃. In some embodiments, Y is O, Z is O, A is CH₂CF₃, G is OCH₂CF₃, and X is H. In some embodiments, Y is O, Z is O, A is C₂H₅, G is OC₂H₅, and X is CF₂Br. In some embodiments, Y is O, Z is O, A is C₂H₅, G is OC₂H₅, and X is ON.

In some embodiments of Formula I, Y is O, Z is a bond, A is C₂H₄OH, G is OCH₃, and X is OCH₃.

Some useful compounds of Formula I with biofilm dispersant, biofilm remover, antibiofilm, antifouling, antifungal, and/or antimicrobial activity include compounds having the following structures as shown in Table 1.

TABLE 1

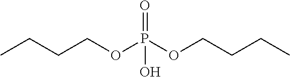

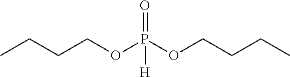

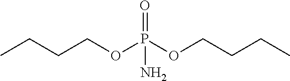

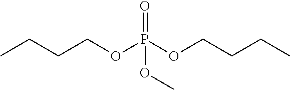

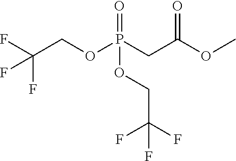 [SMILES: COC(=O)CP(=O)(OCC(F)(F)F)OCC(F)(F)F]

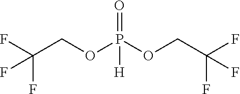 [SMILES: O=[PH](OCC(F)(F)F)OCC(F)(F)F]

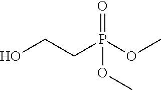

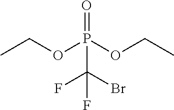

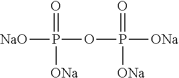

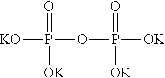

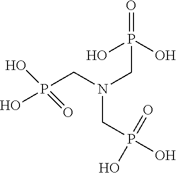 [SMILES: O=P(O)(O)CN(CP(=O)(O)O)CP(=O)(O)O]

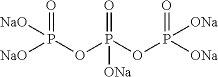

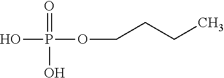

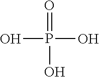

Compounds of Formula I may also include compounds having the following structures as shown in Table 2.

TABLE 2

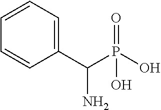 [SMILES: NC(c1ccccc1)P(=O)(O)O]

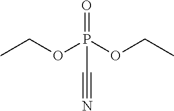

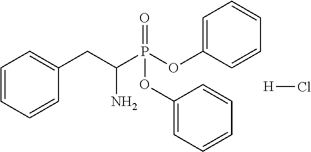

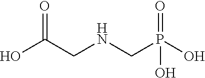

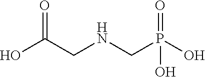

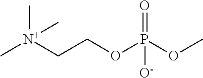

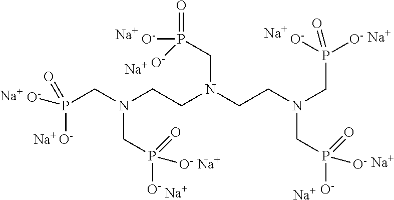

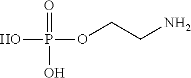

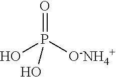

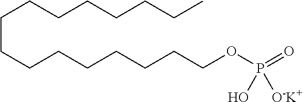

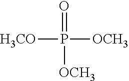

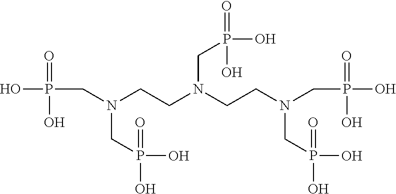 [SMILES: O=P(O)(O)CN(CCN(CP(=O)(O)O)CP(=O)(O)O)CCN(CP(=O)(O)O)CP(=O)(O)O]

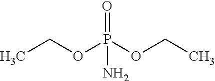

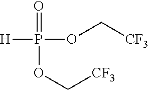 [SMILES: O=[PH](OCC(F)(F)F)OCC(F)(F)F]

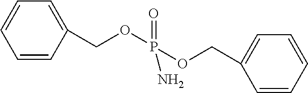 [SMILES: NP(=O)(OCc1ccccc1)OCc1ccccc1]

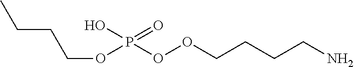

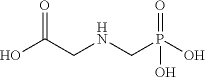

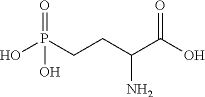 [SMILES: NC(CCP(=O)(O)O)C(=O)O]

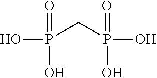

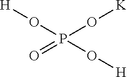

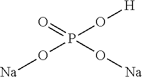

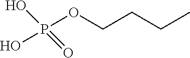

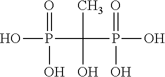

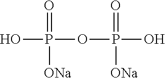

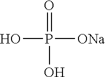

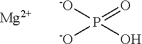

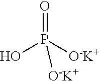

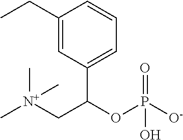 [SMILES: CCc1cccc(C(C[N+](C)(C)C)OP(=O)([O-])O)c1]

Compounds of Formula I may also include compounds having the following structures:

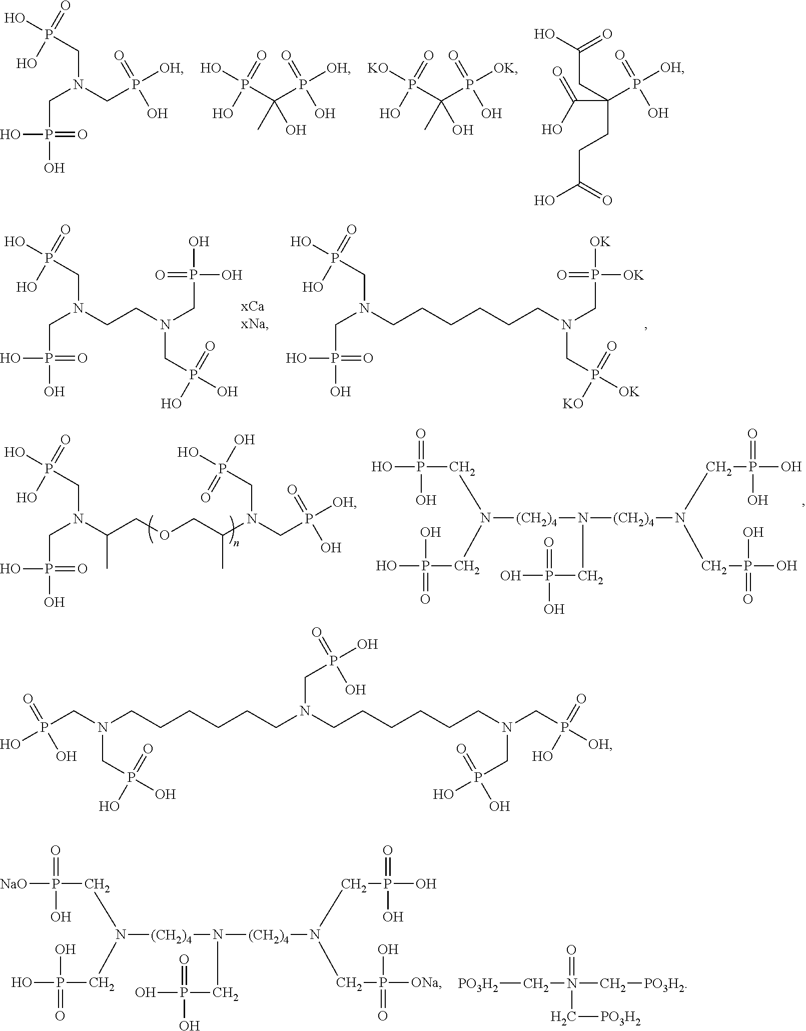

Compounds of Formula I may also include compounds having the following structures, as shown in Table 3.

TABLE 3

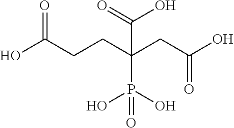 [SMILES: O=C(O)CCC(CC(=O)O)(C(=O)O)P(=O)(O)O]

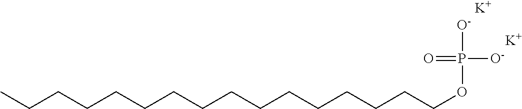 [SMILES: [K+] ; CCCCCCCCCCCCCCCCOP(=O)([O-])[O-] ; [K+]]

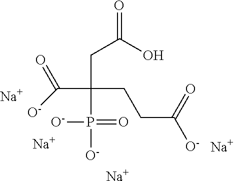 [SMILES: [Na+] ; O=C([O-])CCC(CC(=O)O)(C(=O)[O-])P(=O)([O-])[O-] ; [Na+] ; [Na+] ; [Na+]]

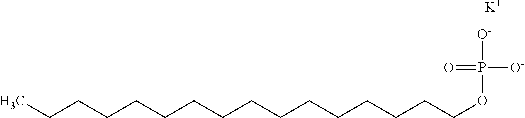 [SMILES: [K+] ; CCCCCCCCCCCCCCCCOP(=O)([O-])[O-]]

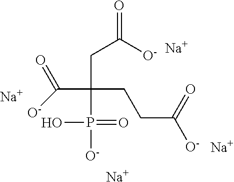 [SMILES: [Na+] ; O=C([O-])CCC(CC(=O)[O-])(C(=O)[O-])P(=O)([O-])O ; [Na+] ; [Na+] ; [Na+]]

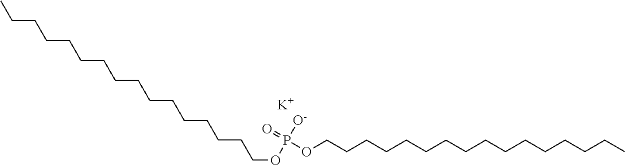 [SMILES: CCCCCCCCCCCCCCCCOP(=O)([O-])OCCCCCCCCCCCCCCCC ; [K+]]

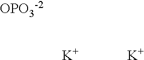 [SMILES: "OPO3-2" ; [K+] ; [K+]]

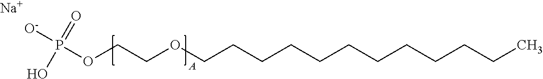

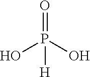

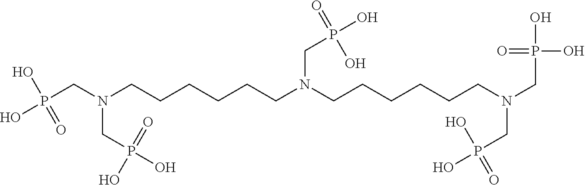 [SMILES: O=P(O)(O)CN(CCCCCCN(CP(=O)(O)O)CP(=O)(O)O)CCCCCCN(CP(=O)(O)O)CP(=O)(O)O]

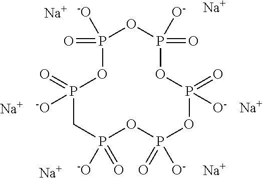

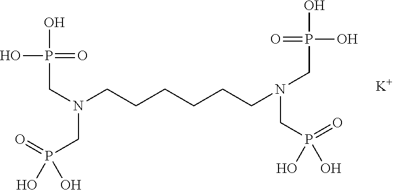 [SMILES: O=P(O)(O)CN(CCCCCCN(CP(=O)(O)O)CP(=O)(O)O)CP(=O)(O)O ; [K+]]

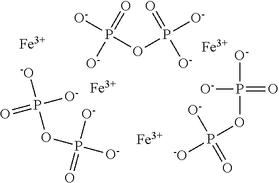

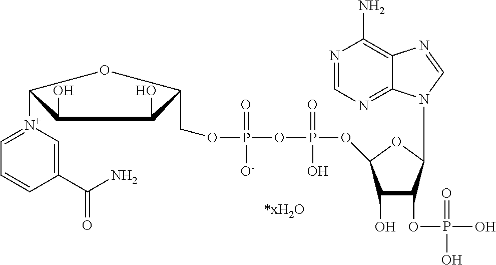

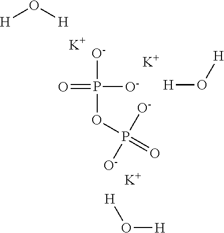

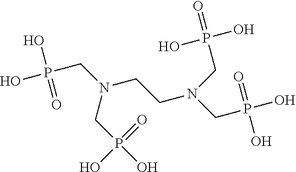 [SMILES: O=P(O)(O)CN(CCN(CP(=O)(O)O)CP(=O)(O)O)CP(=O)(O)O]

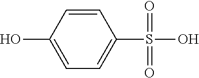 [SMILES: O=S(=O)(O)c1ccc(O)cc1]

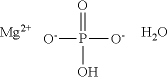

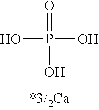

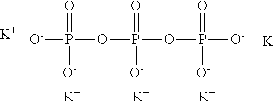 [SMILES: [K+] ; O=P([O-])([O-])OP(=O)([O-])OP(=O)([O-])[O-] ; [K+] ; [K+] ; [K+] ; [K+]]

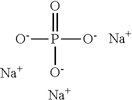

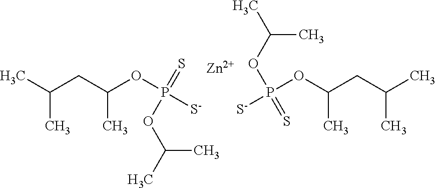

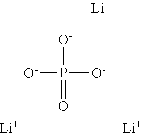

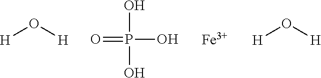

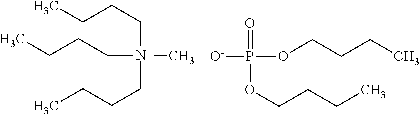

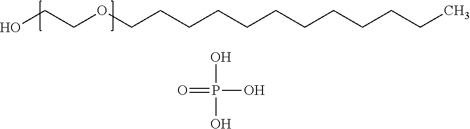

Some useful compounds of Formula II with dispersant, antibiofilm, antifouling, antifungal, and/or antimicrobial activity may include compounds having the following structures as shown in Table 4.

TABLE 4

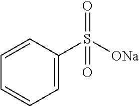

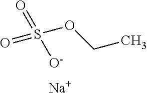

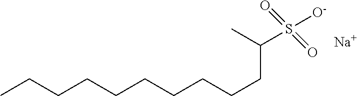 [SMILES: CCCCCCCCCCC(C)S(=O)(=O)[O-] ; [Na+]]

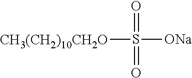

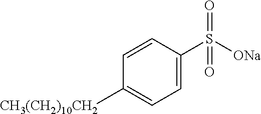

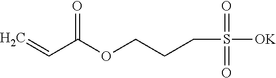

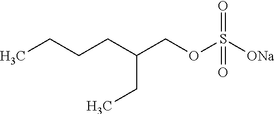

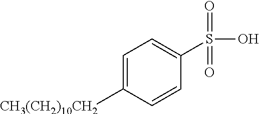

Compounds of Formula II may also include compounds having the following structures, as shown in Table 5.

TABLE 5

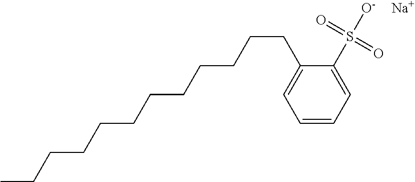 [SMILES: CCCCCCCCCCCCc1ccccc1S(=O)(=O)[O-] ; [Na+]]

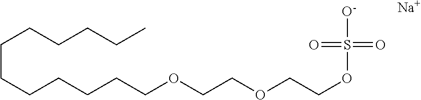 [SMILES: [Na+] ; CCCCCCCCCCCCOCCOCCOS(=O)(=O)[O-]]

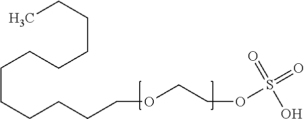

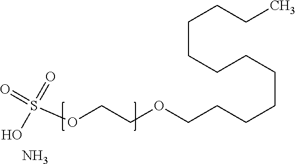

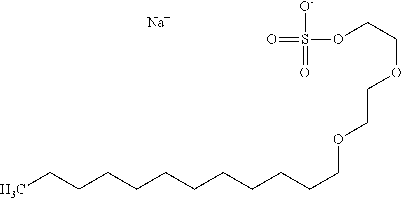 [SMILES: CCCCCCCCCCCCOCCOCCOS(=O)(=O)[O-] ; [Na+]]

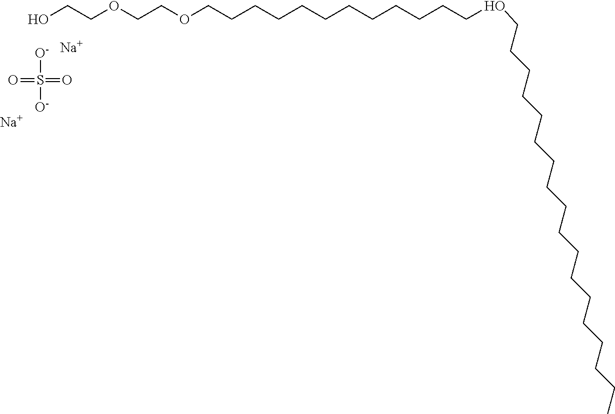 [SMILES: [Na+] ; O=S(=O)([O-])[O-] ; CCCCCCCCCCCCCCCCCCOCCCCCCCCCCCCOCCOCCO ; [Na+]]

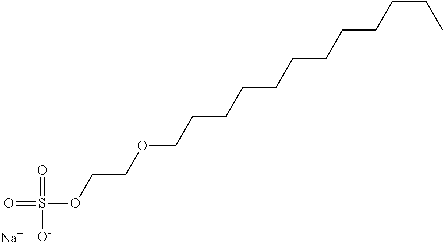 [SMILES: CCCCCCCCCCCCOCCOS(=O)(=O)[O-] ; [Na+]]

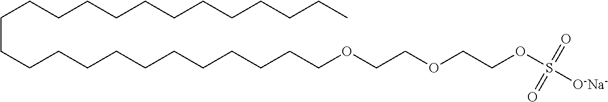

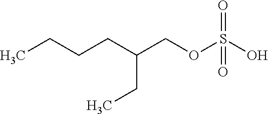

Other compounds useful in the methods of the present disclosure may include compounds having the following structures as shown in Table 6.

TABLE 6

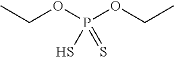

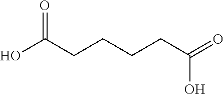

Compounds of Formula I or Formula II (“subject compound”) may be used to disperse, remove, inhibit, reduce, or prevent fungal or microbial growth. For example, a subject compound may be used to reduce or prevent microbial formation of a biofilm. A biofilm includes an assemblage of surface-associated microbial cells that forms on an extracellular surface.

The methods of the present disclosure are methods of dispersing, removing, or inhibiting the growth of a biofilm, or inhibiting the growth of, or killing a fungus or bacteria, comprising contacting the biofilm, fungus or bacteria with an amount of a compound of Formula I, or a composition comprising an amount of a compound of Formula I, wherein the amount of the compound of Formula I is effective to disperse, remove or inhibit the growth of the biofilm, or inhibit the growth of, or kill the fungus or bacteria.

In some embodiments, the methods are methods of dispersing, removing, or inhibiting the growth of a biofilm comprising contacting the biofilm with an amount of a compound of Formula I, or a composition comprising an amount of a compound of Formula I, wherein the amount of the compound of Formula I is effective to disperse, remove, or inhibit the growth of the biofilm.

In some embodiments, the methods comprise contacting the biofilm with an amount of a compound of Formula I effective to disperse, remove, or inhibit the growth of the biofilm.

In some embodiments, the methods comprise contacting the biofilm with a composition comprising an amount of a compound of Formula I effective to disperse, remove, or inhibit the growth of the biofilm.

In some embodiments, the methods are methods of inhibiting the growth of, or killing a fungus or bacteria, comprising contacting the fungus or bacteria with an amount of a compound of Formula I, or a composition comprising an amount of a compound of Formula I, wherein the amount of the compound of Formula I is effective to inhibit the growth of or kill the fungus or bacteria.

In some embodiments, the methods comprise contacting the fungus or bacteria with an amount of a compound of Formula I effective to inhibit the growth of or kill the fungus or bacteria.

In some embodiments, the methods comprise contacting the fungus or bacteria with a composition comprising an amount of a compound of Formula I effective to inhibit the growth of or kill the fungus or bacteria.

The methods of the present disclosure are methods of dispersing, removing, or inhibiting the growth of a biofilm, or inhibiting the growth of, or killing a fungus or bacteria, comprising contacting the biofilm, fungus or bacteria with an amount of a compound of Formula II, or a composition comprising an amount of a compound of Formula II, wherein the amount of the compound of Formula II is effective to disperse, remove, or inhibit the growth of the biofilm, or inhibit the growth of, or kill the fungus or bacteria.

In some embodiments, the methods are methods of dispersing, removing, or inhibiting the growth of a biofilm comprising contacting the biofilm with an amount of a compound of Formula II, or a composition comprising an amount of a compound of Formula II, wherein the amount of the compound of Formula II is effective to disperse, remove, or inhibit the growth of the biofilm.

In some embodiments, the methods comprise contacting the biofilm with an amount of a compound of Formula II effective to disperse, remove, or inhibit the growth of the biofilm.

In some embodiments, the methods comprise contacting the biofilm with a composition comprising an amount of a compound of Formula II effective to disperse, remove, or inhibit the growth of the biofilm.

In some embodiments, the methods are methods of inhibiting the growth of, or killing a fungus or bacteria, comprising contacting the fungus or bacteria with an amount of a compound of Formula II, or a composition comprising an amount of a compound of Formula II, wherein the amount of the compound of Formula II is effective to inhibit the growth of or kill the fungus or bacteria.

In some embodiments, the methods comprise contacting the fungus or bacteria with an amount of a compound of Formula II effective to inhibit the growth of or kill the fungus or bacteria.

In some embodiments, the methods comprise contacting the fungus or bacteria with a composition comprising an amount of a compound of Formula II effective to inhibit the growth of or kill the fungus or bacteria.

In one embodiment, a compound of Formula I or Formula II is present in a composition at a concentration of from about 0.001 to about 10% by weight, based on 100% total weight of the composition and more preferably from about 0.1 to about 1 or 2% by weight, based on 100% total weight of the composition.

According to another embodiment, the compounds is present at a concentration of from about 10-30% by weight, based on 100% total weight of the composition.

According to another embodiment, the compounds is present at a concentration of from about 5-85% by weight, based on 100% total weight of the composition.

Generally, the compounds of the present invention reduce the number of microorganisms (bacteria, fungi, and/or algae) by 95, 99, 99.9, or 99.99% typically within an hour and maintains efficacy over long periods of time.

The compounds of the present invention prevent and/or remove bacterial, fungal, and/or algae biofilm by 95, 99, 99.9, or 99.99% typically within 10 minutes and maintains efficacy over long periods of time.

The compounds of the present invention disperse particles by 45-100% within 30 minutes, dependent on the size of the particle, and maintains efficacy over long periods of time.

Biofilms may form on a wide variety of surfaces, including living tissues, indwelling medical devices, industrial or potable water system piping, or natural aquatic systems. Subject compounds can be used as emulsifiers, dispersants, surfactants, or antifungal, antibiofilm, antifouling, antibacterial or bactericidal agents to remove disease-causing organisms from external surfaces, including plant, human and animal tissue such as skin and wounds. They can be used in different products such as soaps, detergents, deodorizers, stain removers, health and skincare products, cosmetics, antiseptics, and household, industrial, institutional, and clinical cleaners. They can also be used to remove algae, fungi, mold, or slime. Subject compounds can be used alone, or in combination with other antimicrobial or antifungal agents.

A spectrum of indwelling medical devices (e.g., ocular lenses, dental implants, central venous catheters and needleless connectors, endotracheal tubes, intrauterine devices, mechanical heart valves, coronary stents, vascular bypass grafts, pacemakers, peritoneal dialysis catheters, prosthetic joints, central nervous system shunts, tympanostomy tubes, urinary catheters, and voice prostheses) or other devices used in the health-care environment have been shown to harbor biofilms, resulting in measurable rates of device-associated infections.

The subject compounds can be used on the surface of or within medical devices to provide long term protection against bacterial colonization and reduce the incidence of device-related infections. These substances can also be incorporated as an anti-biofilm forming agent, in combination with an antibiotic, into coatings for indwelling medical devices, instruments, and other clinical surfaces. Coatings will sufficiently kill or inhibit the initial colonizing bacteria or fungi and prevent device-related infection as long as the substance is presented in an inhibitory concentration at the device-microbe interface.

The subject compounds, either administered alone or as part of a coating or medical device, can reduce or prevent biofilms. In certain embodiments, biofilms are reduced by about 1.0 log, about 1.5 logs, about 2.0 logs, about 2.5 logs, about 3.0 logs, about 3.5 logs, about 4.0 logs, about 4.5 logs, or about 5.0 logs, or by any number bound by the range of about 1.0 to about 5.0 logs.

The medical devices which are amenable to coatings of the subject anti-biofilm substances generally have surfaces composed of thermoplastic or polymeric materials such as polyethylene, Dacron, nylon, polyesters, polytetrafluoroethylene, polyurethane, latex, silicone elastomers and the like. Devices with metallic surfaces are also amenable to coatings with the anti-biofilm substances. Such devices, for example bone and joint prosthesis, can be coated by cement mixture containing the subject anti-biofilm substances. During implant use, the anti-biofilm substances leach from the cement into the surrounding prosthesis surface environment.

Various methods can be employed to coat the surfaces of medical devices with the anti-biofilm substances. For example, one of the simplest methods would be to flush the surfaces of the device with a solution of the anti-biofilm substance. The flushing solution would normally be composed of sterile water or sterile normal saline solutions. Another method of coating the devices would be to first apply or adsorb to the surface of the medical device a layer of tridodecylmethyl ammonium chloride (TDMAC) surfactant followed by a coating layer of anti-biofilm substance. For example, a medical device having a polymeric surface, such as polyethylene, silastic elastomers, polytetrafluoroethylene or Dareen, can be soaked in a 5% by weight solution of TDMAC for 30 minutes at room temperature, air dried, and rinsed in water to remove excess TDMAC. Alternatively, TDMAC precoated catheters are commercially available; for example, arterial catheters coated with TDMAC are available from Cook Critical Care, Bloomington, Ind. The device carrying the absorbed TDMAC surfactant coated can then be incubated in a solution of the anti-biofilm substance for one hour or so, washed in sterile water to remove unbound anti-biofilm substance and stored in a sterile package until ready for implantation. A further method useful to coat the surface of medical devices with the subject antibiotic combinations involves first coating the selected surfaces with benzalkonium chloride followed by ionic bonding of the anti-biofilm substance composition. Alternative methods and reagents provided in U.S. Pat. Nos. 4,107,121, 4,442,133, 4,678,660 and 4,749,585, 4,895,566, 4,917,686, 4,952,419, and 5,013,30, can be used to coat devices with the anti-biofilm substances disclosed herein.

A subject compound can be directly incorporated into the polymeric matrix of the medical device at the polymer synthesis stage or at the device manufacture stage. A subject compound can also be covalently attached to the medical device polymer.

Biofilms in industrial systems cause severe clogging, contamination, corrosion, scale, slime, fouling, and biodeterioration. Bacterial contamination of the water distribution systems can occur if biofilms are sloughed off naturally or removed by treatment. Biofilms in drinking water piping systems accommodate Escherichia coli, Helicobacter pylon, Mycobacterium spp., and protozoa infected with Legionella pneumophila. This results in decreased water quality and increased treatment costs and health risks. Biofilms in pipes carrying water or other liquids cause reduced flow and increased resistance to flow. Formation of biofilms on probes, sensors, screens and filters results in reduced efficiency. Microbial films that grow on the walls of heat exchanger tubes create additional heat transfer and fluid flow resistances. Formation of biofilms on ship hulls leads to biofouling resulting in increased fuel consumption and cleaning costs. The food industry is also affected by the contamination caused by these films which adhere easily to the walls of food processing equipment. Biofilms in cooling towers results in reduced performance, degradation of material and also provides a reservoir for pathogens. Building materials such as stone, bricks and concrete or clay based roof tiles, mortars and especially all new materials for insulation and damming of humidity often contain organic compounds and are very susceptible to growth of sub-aerial biofilms creating an anaesthetic biopatina and reducing durability. Chemical and physical biodeteriorative forces, phenomena and processes further create damage on old and new buildings. Depending on the environmental conditions water retention and penetration the surface biofilms may transform into networks going deeper into the material. Biocide impregnation of new materials and biocide treatments of monuments create health and environmental hazards.

Subject compounds can be used as antibiofilm agents in industrial systems.

Fouling is an undesirable growth of biological material on a surface immersed in water. Fouling usually starts with adhering and spreading of populations of bacteria over surfaces that are in contact with water. The bacteria pioneers are followed by numerous different algae, invertebrate larvae, hydroids, bryozoans, sponges, tunicates, echinoderms, cnidarians, and coelenterates.

Marine fouling occurs not only on marine vessels such as ships' hulls and drive systems, but also on other structures exposed to water. Such structures may include: pilings, marine markers, undersea conveyances like cabling and pipes, fishing nets, bulkheads, cooling towers, and any device or structure that operates submerged.

A subject compound can be incorporated into marine coatings to limit undesirable marine fouling. The antifouling coatings of this disclosure offer significant advantages over previous attempts to solve marine fouling problems. The coatings disclosed herein can be formulated so as not to contain toxic materials (such as heavy metals), and still retain their efficacy. This avoids the environmental concerns associated with the use of heavy metal biocides.

In certain embodiments, a subject compound is incorporated into an antifouling paint. Antifouling paints comprising a subject compound may further contain binders(s), pigment(s), solvent(s) and additive(s). Solvents can carry the solid components of paint and may be used to obtain the desired viscosity and correct consistency. Examples of the solvent include, but are not limited to, aromatic hydrocarbons such as xylene and toluene; aliphatic hydrocarbons such as hexane and heptane, esters such as ethyl acetate and butyl acetate; amides such as N-methylpyrrolidone and N,N-dimethylformamide; alcohols such as isopropyl alcohol and butyl alcohol; ethers such as dioxane, THF and diethyl ether; and ketones such as methyl ethyl ketone, methyl isobutyl ketone and methyl isoamyl ketone. The solvent may be used alone or in combination thereof.

The binder or resin is the basic solid film former that remains after the solvent has evaporated and may bind the pigment particles together into a cohesive paint film. The binder determines many of the necessary film properties such as adhesion, gloss level, hardness, abrasion resistance, flexibility, speed of drying and durability. Examples of binders include, but are not limited to, alkyd resin, acrylic or vinyl emulsions, polyurethane resins, epoxy resins, silicone based resins, acrylic resins and inorganic silicate based resins. Among the binders which have been used in antifouling coatings are vinyl resins, particularly a vinyl chloride/vinyl acetate copolymer, and rosin.

The paint composition can contain one or more pigments. The pigments used in paint may be present as fine solid particles that are dispersed, but not soluble, in the binder and solvent. Examples of pigments include, but are not limited to, titanium dioxide, cuprous oxide, iron oxide, talc, aluminum flakes, mica flakes, ferric oxide, cuprous thiocyanate, zinc oxide, cupric acetate meta-arsenate, zinc chromate, zinc dimethyl dithiocarbamate, zinc ethylene bis(dithiocarbamate) and zinc diethyl dithiocarbamate.

Additive ingredients may optionally be incorporated into a coating composition. Examples of the additive ingredients are dehumidifiers, wetting/dispersing agents, anti-settling agents, anti-skinning agents, drying/curing agents, anti-marring agents and additives ordinarily employed in coating compositions as stabilizers and anti-foaming agents. Also, any antibiotic which is toxic to gram negative organisms and which is relatively insoluble in seawater can be used with an antifouling marine paint.

The antifouling coatings so produced can be used for the submersible surfaces of boat hulls, pilings, buoys, floating or emplaced offshore platforms, submergence vehicles, navigational aids, aquaculture netting, gear, and equipment, energy technologies, including current, wave, tidal, and other water hydrodynamic technologies, thermal energy technologies, water intake pipes, open an closed water systems, including for irrigation, cooling towers, pumps, reverse osmosis filters and membranes, and any structures or surfaces in contact with fresh or salt water where biofouling maybe a problem.

A subject compound may be used as an emulsifier, particle dispersant, surfactant, or cleaning product.

In some embodiments, the methods of the present disclosure comprise applying to a surface a composition comprising an amount of a compound of Formula I effective to inhibit the growth of a biofilm on the surface. In some embodiments, the surface is an indwelling medical device. In some embodiments, the surface is a surface exposed to water. In some embodiments, the surface is a piece of industrial equipment. In some embodiments the surface is a marine vessel.

In some embodiments, the methods of the present disclosure comprise applying to a surface a composition comprising an amount of a compound of Formula I effective to inhibit the growth of a biofilm on the surface, wherein the composition is an antifouling paint or coating. In some embodiments, the antifouling paint or coating composition further comprises a binder, a pigment, a solvent, or an additive.

In some embodiments, the methods of the present disclosure comprise applying to a surface a composition comprising an amount of a compound of Formula I effective to inhibit the growth of a bacteria or a biofilm on the surface, wherein the composition is an antibacterial soap, an antibacterial detergent, an antibacterial health and skincare product, or an antibacterial household cleaning product.

In some embodiments, the methods of the present disclosure comprise applying to a surface a composition comprising an amount of a compound of Formula II effective to inhibit the growth of a biofilm on the surface. In some embodiments, the surface is an indwelling medical device. In some embodiments, the surface is a surface exposed to water. In some embodiments, the surface is a piece of industrial equipment. In some embodiments the surface is a marine vessel.

In some embodiments, the methods of the present disclosure comprise applying to a surface a composition comprising an amount of a compound of Formula II effective to inhibit the growth of a biofilm on the surface, wherein the composition is an antifouling paint or coating. In some embodiments, the antifouling paint or coating composition further comprises a binder, a pigment, a solvent, or an additive.

In some embodiments, the methods of the present disclosure comprise applying to a surface a composition comprising an amount of a compound of Formula II effective to inhibit the growth of a bacteria or a biofilm on the surface, wherein the composition is an antibacterial soap, an antibacterial detergent, an antibacterial health and skincare product, or an antibacterial household cleaning product.

### Example

Antibacterial and Anti-Biofilm Activity of Compounds

Antibacterial and antibiofilm assay were performed on Staphylococcus aureus (ATCC 25923 and ATCC 12600), S. epidermidis (ATCC 12228 and ATCC 14990), Pseudomonas aeruginosa (ATCC 27853), E. coli (ATCC 25922), C. albicans (ATCC 18804), mixed species oral flora bacteria Streptococcus mutans (ATCC 25175), S. gordonii (ATCC 33399), Aspergillus brasiliensis (ATCC 16404), and/or marine biofilm former Cobetia marina (ATCC 25374).

Antibacterial assays were performed on planktonic bacteria to determine the Minimum Inhibitory Concentrations (MICs) and Minimum Bactericidal Concentrations (MBCs) for the compounds. MICs are defined as the lowest concentration of an antimicrobial that will inhibit the visible growth of a microorganism after overnight incubation. The MICs were determined using the standard CLSI Method M07-A9 for Dilution Antimicrobial Susceptibility Tests for Bacteria That Grow Aerobically modified for 96 well plates. MBCs are the lowest concentration of antimicrobial that kill the organism. MBCs were determined using the standard CLSI Method M26-A for Determining Bactericidal Activity of Antimicrobial Agents modified for 96 well plates.

Antibiofilm assays were performed to determine the prevention and removal of biofilm. The 96-well microplate assay was used to determine biofilm inhibition and the ASTM E2799-12 Standard Test Method for Testing Pseudomonas Biofilm using the MBEC Assay was used to determine biofilm removal. The Minimum Biofilm Eradicating Concentration (MBEC) is defined as the lowest concentration of compound that will eradicate the biofilm. The qualitative MBEC is determined using a microplate reader at absorbance 630 nm and the quantitative MBEC is determined using logo reduction. Clear wells (A₆₃₀<0.1) are evidence of eradication.

Compounds tested in the above assays and some representative results are shown in Table 7.

TABLE 7
Summary of antibacterial and antibiofilm activity of compounds against
S. aureus ATCC 25923

| | | | | MBEC |
|---|---|---|---|---|
| | MIC | Biofilm | Biofilm | Qualitative |
| Compound | (%) | Prevention | Removal | (%) |
| Butyl acid phosphate and | 1.16 | 99.99% | 99.99% | 1.2 |
| Dibutyl phosphate (50:50) | | | | |
| Dibutyl phosphate | 0.24 | 99.99% | 99.99% | 0.24 |
| Dibutyl phosphate | 0.01 | 99.99% | 99.99% | 0.24 |
| Dibutyl phosphoramidate | 1.0 | 99.99% | 99.99% | 0.24 |
| Dibutyl methyl phosphate | 0.24 | 99.99% | 99.99% | >2.3 |

MIC = minimum inhibitory concentration;
MBEC = minimum biofilm eradicating concentration.

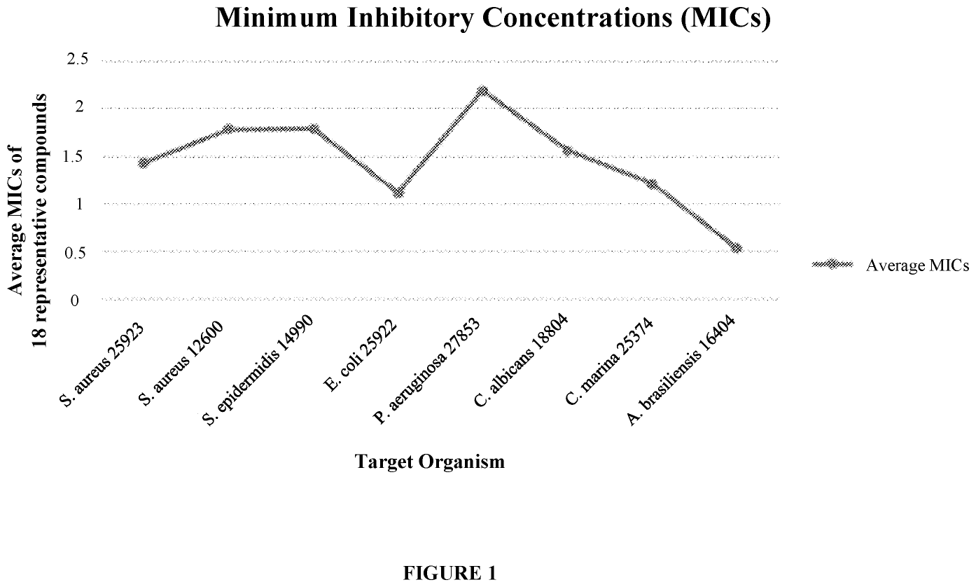

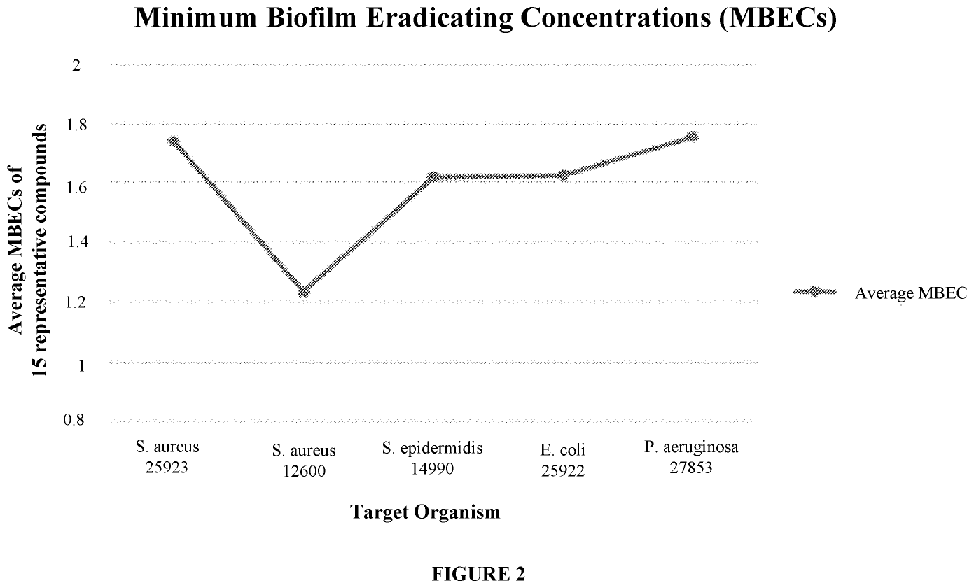

In addition to the above table, the average MICs and MBECs of representative compounds against the organisms tested are illustrated in FIG. 1 and FIG. 2, respectively.

FIG. 1 depicts the Minimum Inhibitory Concentrations (MICs) of 18 organophosphorus and organosulfurous representative compounds against Gram-positive and Gram-negative bacteria and fungi. Clear wells (A₆₃₀<0.1) are evidence of inhibition.

FIG. 2 depicts the Minimum Biofilm Eradicating Concentrations (MBECs) of 15 organophosphorous and organosulfurous representative compounds against Gram-positive and Gram-negative bacteria. Clear wells (A₆₃₀<0.1) are evidence of eradication.

In brief, the tested compounds inhibited the growth of the target microorganisms 99.99% (Table 7; FIG. 1). The tested compounds prevented biofilm formation by the target microorganisms up to 99.99% (p<0.01). The tested compounds removed existing biofilm formed by the target microorganisms 99.99% (Table 7; FIG. 2). Quantitative MBEC log₁₀ reduction ranged from 3.9 to 4.4. Table 7 shows a summary of antibacterial and antibiofilm activity of tested compounds against S. aureus ATCC 25923. The activity of the tested compounds against the other target biofilm formers showed similar results.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1-48. (canceled)

49. A method of dispersing or removing a biofilm and/or killing fungi or bacteria in a biofilm, comprising contacting the biofilm with an amount of a compound or a salt thereof, or a composition comprising an amount of a compound or a salt thereof, wherein the amount of the compound is effective to disperse or remove the biofilm, or kill fungi or bacteria in the biofilm, wherein the compound has the structure: 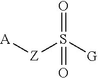
wherein A is H, C₁₋₂₀ hydrocarbyl, alkylaryl, or optionally R¹ substituted C₁₋₂₀ hydrocarbyl; Z is O or a bond; G is OH, H, C₁₋₆—COO-alkyl, —O—C₁₋₆ alkyl, R¹ substituted C₁₋₁₀ hydrocarbyl, CH₂NHCH₂COOH, or O-aryl;
R¹ is selected from halogen, cyano, OH, C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, and NR³CONR⁴, wherein each of C₁₋₆ hydrocarbyl, C₁₋₆ alkoxy, SOR², SO₂R², SO₂NR³R⁴, CONR³R⁴, NR³R⁴, NR³COR⁴, NR³SO₂R⁴, NR³CO₂R⁴, NR³CONR⁴, can be optionally substituted with halo, amino, hydroxyl, C₁₋₆ hydrocarbyl, C₁₋₆ alkoxy, or cyano;
R², R³ and R⁴ are independently selected from hydrogen, C₁₋₆ hydrocarbyl, in which each of the C₁₋₆ hydrocarbyl can be optionally substituted with halo, amino, hydroxyl, C₁₋₆ alkoxy, or cyano.

50. The method of claim 49, wherein the compound or a salt thereof is selected from the group consisting of:

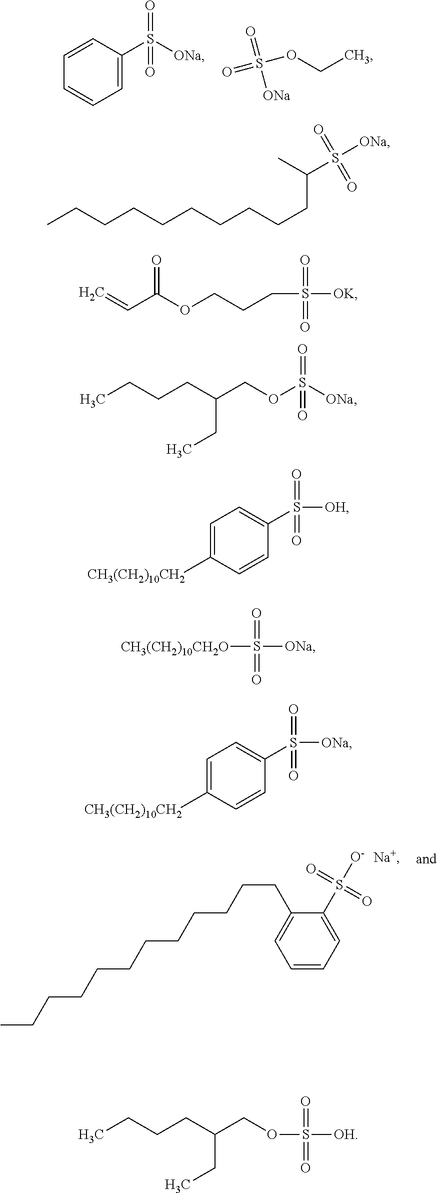

51. The method of claim 49, comprising applying to a surface the compound or a salt thereof or the composition to disperse or remove the biofilm on the surface or kill bacteria or fungi in the biofilm on the surface.

52. The method of claim 51, wherein the surface comprises human or animal tissue, an indwelling medical device, a surface exposed to water, a piece of industrial equipment, or a marine vessel.

53. The method of claim 52, wherein the compound or salt thereof or the composition is in an antifouling paint or coating and optionally wherein the antifouling paint or coating further comprises a binder, a pigment, a solvent, or an additive.

54. The method of claim 49, wherein the compound or salt thereof or the composition comprises the compound having the structure:

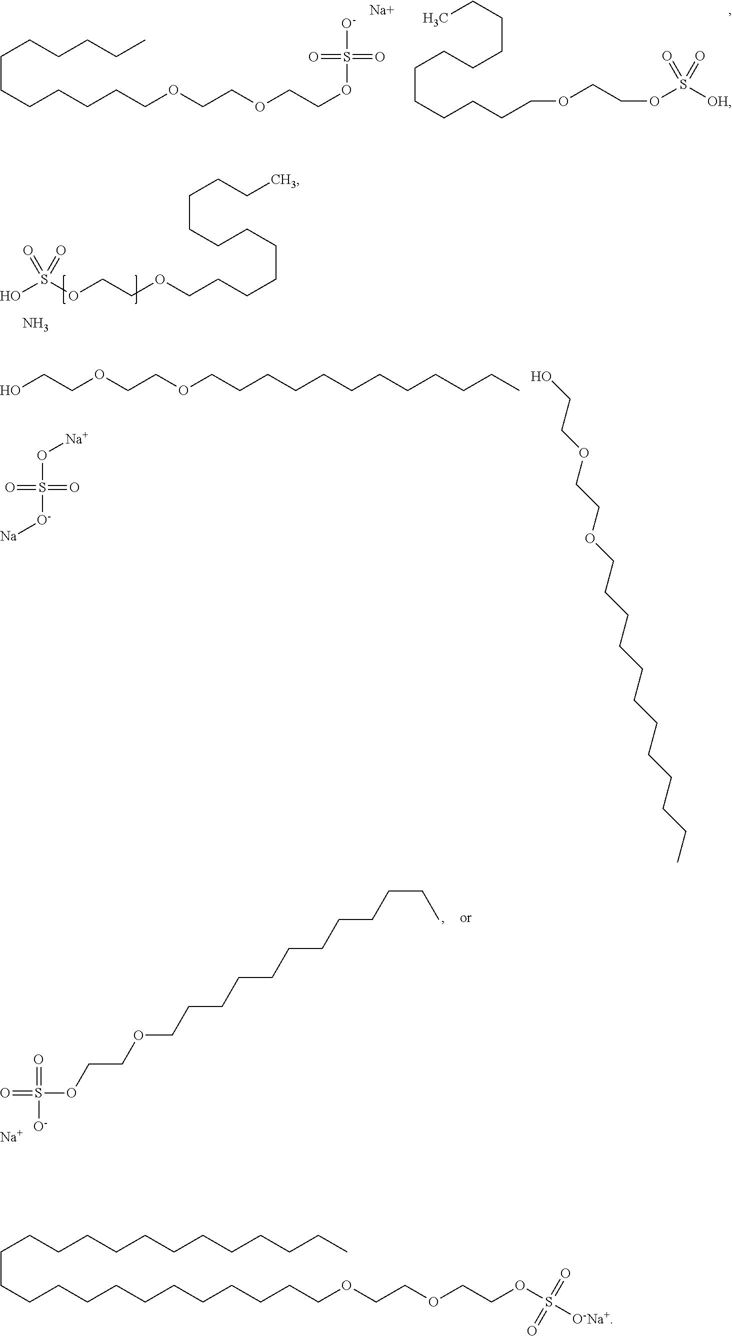

55. The method of claim 49, wherein the compound or salt thereof is in an antibacterial or antifungal soap, an antibacterial or antifungal detergent, an antibacterial or antifungal health and skincare product, an antibacterial household or industrial cleaning product, a stain remover, or an antiseptic.

56. The method of claim 49, wherein the composition comprises an antibacterial or antifungal soap, an antibacterial or antifungal detergent, an antibacterial or antifungal health and skincare product, an antibacterial household or industrial cleaning product, a stain remover, or an antiseptic.

57. The method of claim 49, wherein the method (i) disperses or removes a biofilm and (ii) kills fungi or bacteria in a biofilm.

58. The method of claim 57, wherein the method further inhibits growth of and/or kills fungi or bacteria.

59. The method of claim 49, wherein the method (i) disperses or removes a biofilm and (ii) inhibits growth of and/or kills fungi or bacteria.

60. The method of claim 50, wherein the method (i) kills fungi or bacteria in biofilm and (ii) inhibits growth of and/or kill fungi or bacteria.

61. The method of claim 49, wherein the method (i) disperses or removes a biofilm or (ii) kills fungi or bacteria in a biofilm.
